(19) United States
(12) Patent Application Publication
(10) Pub. No.: US 2007/0250109 A1
(43) Pub. Date: Oct. 25, 2007

(54) Tourniquets

(75) Inventors: Ryan Kerstein, London (GB); Christian Fellows, London (GB)

Correspondence Address: SALTAMAR INNOVATIONS, 30 FERN LANE, SOUTH PORTLAND, ME, 04106, US

(73) Assignee: IMPERIAL INNOVATIONS, London (GB)

(21) Appl. No.: 11/573,553

(22) PCT Filed: Aug. 10, 2005
(86) PCT No.: PCT/EP05/53950

§ 371 Date: Jun. 22, 2007

(30) Foreign Application Priority Data
Aug. 12, 2004 (GB) 0417949.5

(51) Int. Cl. A61B 17/00

(52) U.S. Cl. 606/203

(57) ABSTRACT

A disposable tourniquet is disclosed which comprises a band (10) of material having an aperture (16) along its length. A grip region (18) is disposed between the aperture (16) and one end (12) of the band. A fastening region (14) sufficiently narrow to pass through the aperture (16), is arranged at or near the other end of the band and is separated from the aperture (16) by a loop region having sufficient length to encircle a limb. An adhesive (22) serves to stick overlapping regions of the band (10) to one another after the loop region (20) has been wrapped around a limb and the fastening region (14) has been passed through the aperture (16).

### FIELD OF THE INVENTION

This invention relates to a disposable tourniquet.

### BACKGROUND OF THE INVENTION

The use of tourniquets in a clinical environment is primarily for the purpose of limiting venous blood flow in a limb to ensure vessel distension, to assist in venepuncture and cannulation procedures, also referred to as blood work. There are currently two types of tourniquets in common use, namely reusable and disposable. Re-usable tourniquets, the most common having the form of a length of elasticated fabric, provide excellent performance in terms of their ease of use and patient comfort. However, studies have shown that the re-use of a tourniquet poses a risk of infections being passed from one patient to another.

In order to overcome this problem, various disposable tourniquets have been developed. However, while reducing the risk of cross-infection, such tourniquets have not performed as well as re-usable tourniquets and they can be uncomfortable for patients.

U.S. Pat. No. 5,219,356 describes a disposable tourniquet comprising an elongated, flat, stretchable band. The band has a pressure sensitive adhesive face on one side of the band at one end, and a release agent on the same face of the band but spaced from the adhesive face. The tourniquet is stored with the adhesive face folded against and adhering to the release agent and is deployed by peeling the adhesive face away from the release agent, wrapping the tourniquet about the arm and pulling it sufficiently tight and then adhering the adhesive face of the outside surface of the band to hold the tourniquet in place.

It is a disadvantage of this known tourniquet that it is difficult to set the correct tension before the adhesive face is stuck down. The user has to stretch the tourniquet to the extent necessary to achieve the correct tension before adhering the free end of the tourniquet to the band surrounding the patient's arm. While performing this task, the user is also hampered by only being able to grip one end of the band, the other end being trapped beneath the loop encircling the patient's arm. If the tension should prove to be insufficient then the veins of the patient will not be sufficiently distended and if the tension is excessive then the patient can be caused unnecessary discomfort.

### OBJECT OF THE INVENTION

The present invention seeks therefore to provide a disposable tourniquet which makes it easier to set the correct tension while minimising patient discomfort.

### SUMMARY OF THE INVENTION

According to a first aspect of the present invention, there is provided a disposable tourniquet comprising a band of material having an aperture, a grip region disposed between the aperture and one end of the band, a fastening region sufficiently narrow to pass through the aperture, a loop region having sufficient length to encircle a limb and disposed between the aperture and the fastening region, and an adhesive for adhering overlapping regions of the band to one another after the loop region has been wrapped around a limb and the fastening region has been passed through the aperture.

The band may be fabricated from a single sheet material or as composite material to provide the desired characteristics, in particular strength and elasticity. The materials used may be synthetic or natural including a variety of flexible, planar composite fabric materials, which may be woven, knitted or non-woven.

It is preferred for the band to be made of a substantially inextensible material. Plasticised paper, as used in certain mailing envelopes, has been found to be particularly suitable and inexpensive.

According to a second aspect of the present invention there is provided a strip comprising a plurality of disposable tourniquets, each of which tourniquets comprises a band of material characterised by:

an aperture,

a grip region disposed between the aperture and one end of the band,

a fastening region sufficiently narrow to pass through the aperture,

a loop region having sufficient length to encircle a limb and disposed between the aperture and the fastening region, and

an adhesive for adhering overlapping regions of the band to one another after the loop region has been wrapped around a limb and the fastening region has been passed through the aperture,

the tourniquets being detachably connected to one another end to end to form a continuous strip.

It is advantageous to form the tourniquet as part of an elongate continuous strip within which individual tourniquets are detachably connected to one another end to end.

Such a continuous strip can conveniently be formed into a roll and packaged in a dispenser from which tourniquets can be drawn out, one at a time. In this way, it is possible to ensure that each tourniquet remains sterile until the time that it is dispensed.

According to a third aspect of the present invention, there is provided a method for applying a tourniquet to a limb of a patient, the tourniquet comprising:

an aperture,

a grip region disposed between the aperture and one end of the band,

a fastening region sufficiently narrow to pass through the aperture,

a loop region having sufficient length to encircle a limb and disposed between the aperture and the fastening region, and

an adhesive for adhering overlapping regions of the band to one another after the loop region has been wrapped around a limb and the fastening region has been passed through the aperture,

the method comprising the steps of:
i) wrapping the loop region of the tourniquet around the limb;
ii) threading the fastening region through the aperture;
iii) pulling on the grip region and the fastening region in order to tension the loop region of the tourniquet to a point where a required distension of one or more veins in the limb occurs;
iv) placing the fastening region onto the adhering region, whereby the fastening region adheres to the adhesive region to hold the tourniquet in place at a level of tension which is the minimum tension necessary to achieve the required distension of the one or more veins in the limb.

The length of the grip region between the aperture and the end of the band should be sufficient to ensure that it can be gripped firmly while the band is being tensioned around the limb of a patient. The dimensions of the aperture must be sufficient to allow the tensioning region to pass through it while leaving sufficient material around the aperture to ensure that the band does not tear while it is being tensioned.

The adhesive used to secure the tourniquet after it has been wrapped round a limb need not be particularly strong as it only has to withstand shearing forces, there being no tendency for the ends of the tourniquet to peel apart. Consequently, it is possible to use a pressure sensitive adhesive as used, for example, in mailing envelopes. An advantage of this is that after any blood work has been terminated, it is possible to remove the tourniquet easily by using a peeling action to separate the regions adhering to one another.

A protective liner coated with a release agent may be used to cover the adhesive during storage but such a cover may not be required if the tourniquets are wound into a continuous supply roll. The dimensions of a liner, if present, should be at least equal to those of the adhesive but it is preferred to provide a pull tab to ease peeling of the liner away from the adhesive during deployment of the tourniquet.

### BRIEF DESCRIPTION OF THE DRAWINGS

The invention will now be described further, by way of example only, with reference to the accompanying drawings, in which:

FIG. 1 is a plan view of a tourniquet of the present invention, and

FIG. 2 shows the tourniquet of FIG. 1 in the position that it would adopt when wrapped around a limb of a patient.

### DETAILED DESCRIPTION OF THE PREFERRED EMBODIMENT

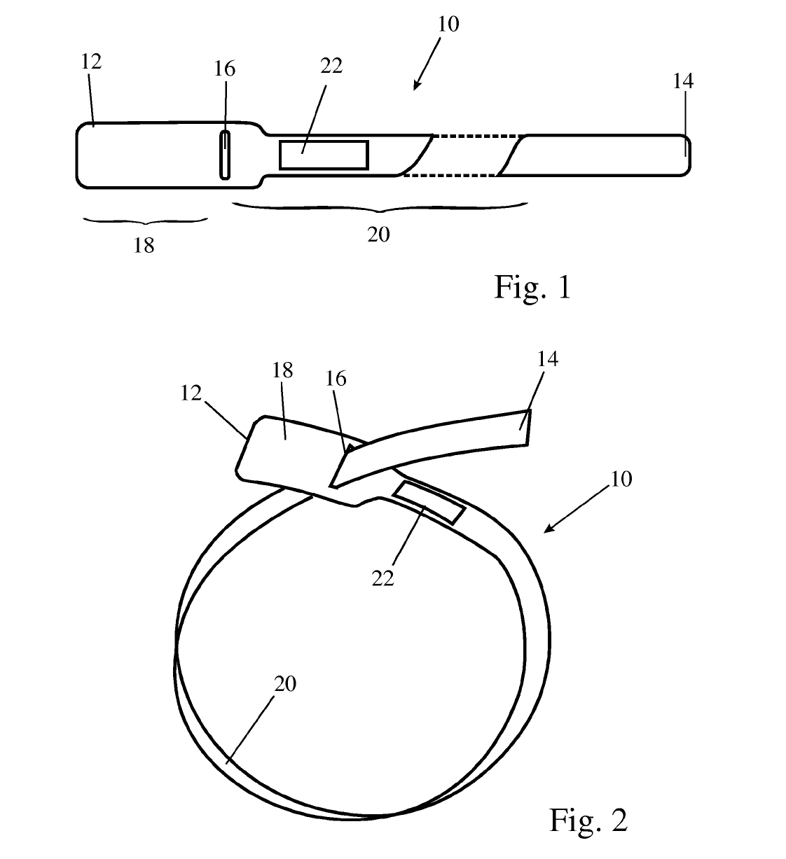

The tourniquet 10 in FIG. 1 is formed as a band of material that is substantially inextensible. The material is preferably a plasticised paper as used in Tyvek® mailing envelopes.

The band has a wider end 12 and a narrower end 14. An aperture 16 is spaced from the wider end 12 by means of a region 18 that acts as a gripping tab and will herein be termed the grip region. To the right of the aperture 16, as viewed, there is provided a region 18 coated with an adhesive. The narrow end 14 of the band 10 is of a sufficient width to pass through the aperture 16 and will herein be termed the fastening region. The region of the band 10 designated 20 in FIG. 1, which extends between the fastening region 14 and the aperture 16 constitutes the loop that is wrapped around the limb of the patient and is herein termed the loop region.

The tourniquet of FIG. 1 is deployed in a manner most readily understood by reference to FIG. 2. The loop region 20 of the band is wrapped around the limb of the patient, usually the upper arm. The fastening region 14 is next threaded through the aperture of 16, as shown in the drawing. By pulling on the grip region 12 and the fastening region 14, it is possible to tension the loop region 20 of the tourniquet to the point where the required distension of the veins occurs. The fastening region 14 may now be brought down and adhered to adhesive region 22 to hold the tourniquet in place.

It is an important advantage of the illustrated tourniquet that it minimises discomfort to the patient. In particular, one can ensure that the correct level of tension has been applied before the ends of the tourniquet are adhered to one another. In this way, it is possible to set the level of tension to the minimum necessary to achieve the required distension of the veins. Furthermore, all parts of the tourniquet used to apply tension around the limb lie in the same plane as one another and there is therefore no tendency to pinch the skin of the patient as the tourniquet is tightened.

The adhesive region 22 need not necessarily be located in the position illustrated. For example, it would be alternatively possible for an adhesive region to be formed on the underside of the grip portion 12 or the fastening portion 14. The adhesive may be permanently exposed or it may be covered with a liner until such time as the tourniquet is to be deployed. Once again this is somewhat analogous to the adhesive found in mailing envelopes.

It is preferred to form the tourniquet 10 as part of a continuous strip wound into a roll and stored within a sterile box, from which only one end of roll protrudes. In this way, it is possible to ensure that the tourniquet remains sterile until such time as it is to be deployed. If tourniquets are packaged as a continuous strip, it is not necessarily to protect the adhesive region 22 with a liner as the turns of the continuous strip will ensure that the adhesive remains covered while it is in storage. Once a tourniquet has been torn off the strip, the adhesive region 22 can be used to stick its ends to each other to hold the tourniquet in place while blood work is being carried out.

After termination of the blood work, the tourniquet can be removed by simply peeling away the fastening region 14 from the adhesive region 22 and pulling on the grip region 12 to release the tourniquet from the patient's limb. The tourniquet can then be discarded to avoid any risk of contaminating another patient.

It should be appreciated that various modifications may be made to the described and illustrated tourniquet without departing from the scope of the invention as set forth in the appended claims. For example, it would be possible for the aperture 16 to be constituted by a recess that reaches as far as the wider end of the band, so long as there remains a region between where the two ends cross over and the wider end of the band, by means of which the band can be gripped during tensioning. The fact that the grip region is split along its length will not interfere with its function. Furthermore, the band has been described as being formed of a single sheet of material but it can be formed of two sheets, one of which has a removable window to expose an adhesive coating on the other.

1-13. (canceled)

14. A disposable tourniquet comprising a band of material having:
an aperture,
a grip region disposed between the aperture and one end of the band,
a fastening region sufficiently narrow to pass through the aperture,
a loop region having sufficient length to encircle a limb and disposed between the aperture and the fastening region, and
an adhesive for adhering overlapping regions of the band to one another after the loop region has been wrapped around a limb and the fastening region has been passed through the aperture,
wherein
the band is made of a substantially inextensible plasticised paper material.

15. A tourniquet according to claim 14, wherein the adhesive is a pressure sensitive adhesive.

16. A tourniquet according to claim 14, wherein the adhesive is covered by means of a protective liner coated with a release agent.

17. A tourniquet according to claim 16, wherein the liner includes a pull tab to ease peeling of the liner away from the adhesive during deployment of the tourniquet.

18. A tourniquet according to claim 14, wherein the aperture extends transversely across the band.

19. A tourniquet according to claim 14, wherein the aperture extends parallel to the length of the band.

20. A tourniquet according to claim 14, wherein the band is formed as part of an elongate continuous strip within which individual tourniquets are detachably connected to one another end to end.

21. A tourniquet according to claim 20, wherein the continuous strip is wound into a roll and packaged in a dispenser from which tourniquets can be drawn out, one at a time.

22. A tourniquet dispenser comprising a strip of inextensible plasticised paper material defining a plurality of disposable tourniquets detachably connected to one another end to end, wherein each tourniquet in the strip comprises
a band having an aperture,
a grip region disposed between the aperture and one end of the band,
a fastening region sufficiently narrow to pass through the aperture,
a loop region having sufficient length to encircle a limb and disposed between the aperture and the fastening region, and
a pressure sensitive adhesive for adhering overlapping regions of the band to one another after the loop region has been wrapped around a limb and the fastening region has been passed through the aperture, and
wherein the strip is wound into a roll and packaged in a container from which tourniquets can be drawn out, one at a time, and separated from the remainder of the roll.

23. A method for applying a tourniquet to a limb of a patient, the tourniquet comprising:
an aperture,
a grip region disposed between the aperture and one end of the band,
a fastening region sufficiently narrow to pass through the aperture,
a loop region having sufficient length to encircle a limb and disposed between the aperture and the fastening region, and
an adhesive for adhering overlapping regions of the band to one another after the loop region has been wrapped around a limb and the fastening region has been passed through the aperture,
the method comprising the steps of:
i) wrapping the loop region of the tourniquet around the limb;
ii)threading the fastening region through the aperture;
iii) pulling on the grip region and the fastening region in order to tension the loop region of the tourniquet to a point where a desired distension of at least one vein in the limb occurs;
iv) placing the fastening region onto the adhering region, whereby the fastening region adheres to the adhesive region to hold the tourniquet in place at a level of tension which is sufficient to achieve the required distension of the vein.
